(19) United States
(12) Patent Application Publication
(10) Pub. No.: US 2010/0274191 A1
(43) Pub. Date: Oct. 28, 2010

(54) METHOD AND APPARATUS FOR INTRA-ARTICULAR INJECTION OR ASPIRATION

(76) Inventor: Choon Meng Ting, Singapore (SG)

Correspondence Address: MICHAUD-Kinney Group LLP, 306 INDUSTRIAL PARK ROAD, SUITE 206, MIDDLETOWN, CT, 06457, US

(21) Appl. No.: 12/747,224

(22) PCT Filed: Jan. 6, 2009
(86) PCT No.: PCT/SG2009/000010

§ 371 Date: Jun. 10, 2010

(30) Foreign Application Priority Data
Jan. 8, 2008 (SG) 200800188-5

(51) Int. Cl. A61M 5/42

(52) U.S. Cl. 604/116

(57) ABSTRACT

A medical apparatus for an intra-articular injection or aspiration, the apparatus having a hollow piercing member through which fluid may pass, the piercing member having a first end adapted to pierce a patient, and a second end releasably secured to a body; a sensing means, wherein upon entering a cavity of the patient the sensor means senses the change in resistance at the first end of the piercing member which results when the first end of the piercing member enters the cavity.

### CROSS-REFERENCE TO RELATED APPLICATIONS

This application is a U.S. National Stage application of International Application Serial No. PCT/SG2009/000010 filed Jan. 6, 2009.

### FIELD OF THE INVENTION

The present invention generally relates to fluid exchange within a joint. In particular the invention relates to a method and apparatus for intra-articular injection or aspiration.

### BACKGROUND ART

Osteoarthritis (arthritis) joints result when the synovial fluid has lost elasticity and viscosity, resulting in a reduction in the lubricating and shock absorbing properties of the synovial fluid. Visco supplementation is a procedure that involves the injection of gel-like substances (hyaluronates) into a joint to supplement the elastic and viscous properties of the synovial fluid. Visco supplementation restores the shock-absorbing and lubricating properties of the synovial fluid in, for example, an arthritic knee joint.

The current procedure requires a health care professional to inject an elastic and viscous fluid that is made from hylans (hyaluronic acid), or equivalent, directly into a patients knee joint three times, 7 days apart, over a 15-day period (days 1, 8, and 15). To get the best results from this therapy, the health care professional may first need to remove the diseased osteoarthritic synovial fluid from the knee before the injection takes place.

Completion of the full three-injection treatment course is necessary to achieve the greatest benefit. In some instances five injections may be given. Additionally removal of the synovial fluid from the osteoarthritic joint should be removed before the new hyaluronic acid is injected. Most patients feel the greatest pain relief 8 to 12 weeks after beginning treatment.

The current procedure involves:

1. Assess initial condition of patient for suitability as recipient of Visco supplementation.

2. The patient is requested to lie down;

3. The knee is bent at a suitable angle (approximately 30 degrees);

4. The knee is checked for the existence of effusion (escape of fluid into a cavity);

5. A sterile field about the joint is prepared (alcohol+iodine);

6. The patella (knee cap) is located as a landmark (see FIG. 1);

7. The needle is positioned at the lateral (see FIG. 3) or medial side of the superior poles of the patella, pointing towards the joint line.

8. As the needle insertion point has been determined, the needle is slowly inserted in the defined area until the practitioner feels the “give” when entering the synovial cavity;

9. Needle is correctly located in the synovial cavity.

Removal of Joint Fluid

10. Any joint fluid that is present is aspirated and disposed.

Visco Supplementation of Synovial Fluid

11. Once the needle is correctly located in the synovial cavity and all joint fluid has been aspirated, the Synovial fluid dosage is injected into the joint cavity (Synovial fluid is a hyaluronan-containing fluid found in a joint that lubricates and cushions the joint).

Post Process

12. Once Visco supplementation has been successfully administered all consumable components are correctly disposed.

13. The needle is removed from the patient.

A barrier to acceptance of this procedure by health care professionals is the perceived difficulty in correctly locating the needle in the synovial cavity. Experienced practitioners develop a proven procedure and “feel” for the correct location and penetration of the needle. A barrier to acceptance of the procedure by health care professionals is the perceived difficulty in correctly locating the needle tip in the synovial cavity.

In the instance the synovial cavity is not correctly located and hyaluronic acid is injected into any other location of the joint a severe hyper immune response will result in the patient's joint, including pain and swelling.

Whilst the above discussion relates to Visco supplementation it is intended to provide a specific example only. Similar problems exist in relation to the injection of fluid into or the aspiration of fluids from a joint.

The preceding discussion of the background to the invention is intended only to facilitate an understanding of the present invention. It should be appreciated that the discussion is not an acknowledgment or admission that any of the material referred to was part of the common general knowledge of at the priority date of the application.

It is an object of this invention to provide a medical apparatus and method of using that apparatus that will assist a practitioner in correctly identifying when a needle tip of the apparatus is located within a patient's cavity.

### SUMMARY OF INVENTION

The present invention provides a medical apparatus for an intra-articular injection or aspiration, the apparatus comprises:

a hollow piercing member through which fluid passes at a constant rate, the piercing member having a first end adapted to pierce a patient, and a second end releasably secured to a body;

a sensing means,

wherein upon entering a cavity of the patient fluid is injected into the patient and the sensor means senses the change in resistance which results when the first end of the piercing member enters the cavity.

Preferably the sensing means is in the form of a pressure sensor. Preferably the sensing means comprises a data logging system that records the pressure profile during the procedure.

The present invention also provides a medical apparatus for an intra-articular injection or aspiration, the apparatus comprises:

a hollow piercing member through which fluid may pass, the piercing member having a first end adapted to pierce a patient, and a second end releasably secured to a body;

a sensing means, wherein upon entering a cavity of the patient the sensing means senses the change in resistance at the first end of the piercing member which results when the first end of the piercing member enters the cavity.

The piercing member may be in the form of a needle or cannula.

The sensing means may be in the form of a pressure sensor capable of detecting fluid flow resistance. The sensing means may sense the resistance to fluid flow relative to the first end of the piercing member. As the first end enters the cavity the resistance at the first end will decrease, allowing fluid to exit the piercing member more easily than when it is passing through other tissues. The decrease in resistance provides an indication to an operator that the first end of the piercing member is in the cavity.

In one aspect of the invention the fluid is injected into the patient at a constant rate.

In another aspect of the invention the body incorporates an inducer to induce pulses within the apparatus, wherein upon entering a cavity of the patient the sensing means senses pulse differentials which result when the first end of the piercing member enters the cavity. The inducer may comprise a pulse mechanism for delivering pulses to the piercing member, whereby the pulses may be measured by the sensor means.

The pulse mechanism delivers pressure pulses to the piercing member. The pulse mechanism applies a suitable pressure pulse to a fluid in the piercing member and the sensing means senses the flow resistance thereto.

The pulse mechanism for providing pulses may comprise a tube within the body of the apparatus, wherein the tube is in fluid communication with the piercing member and contains a fluid therein which is caused to pulse at a predetermined repetition rate and pressure. The fluid may be any saline or anesthetic fluid accepted by the body without causing any excessive response. Isotonic Saline and anesthetic agents are suitable sensing fluids as they do not generally give rise to any reactions. Anesthetic agent can also be useful in reducing the pain of the needle insertion at the same time. The amount used in the pulsing or continuous flow is actually a very minute amount, in terms of a fraction of a ml.

The sensing means may comprise a transducer to measure changes in pressure within the piercing member. Preferably a change in resistance at the first end of the piercing member is detected by a threshold change in the transducer.

Preferably the sensing means comprises a data logging system that records the pressure profile during the procedure.

The apparatus may be connected to a display unit which displays the profile and measurements taken from the transducer and therefore identifies to the operator when the first end of the piercing member enters different regions of the joint.

In another aspect of the invention the sensing means includes a motion sensor to detect the difference in the movement of the piercing member as it passes through material differing in viscosity. The sensing means may amplify the change in reaction force as the first end moves through different tissue.

In this aspect of the invention the pulse mechanism delivers force pulses to the piercing member, whereby the pulses forwardly induce the piercing member whilst the piercing member is being pushed by the operator.

In one aspect of the invention the pulse mechanism is in the form of a mass adapted to move relative to the body in a forward and back direction according to the movement of the apparatus wherein the movement of the mass and its inertial effects deliver the force pulses, inducing the piercing member forward.

The mass may be incorporated within the body.

The sensing means may sense a reduction in inertial kickback through the body of the apparatus.

In another aspect of the invention the pulse mechanism induces force pulses using an eccentrically mounted flywheel.

Preferably the body may be removed from the piercing member and replaced by a syringe.

The apparatus may also comprise a flushing device to clean the cavity into which the piercing member is inserted. The flushing device may be incorporated with the pulse mechanism.

The apparatus may also comprise a visual indication which is highlighted when the sensing means senses that the first end of the piercing member has entered the cavity.

The present invention further provides a needle device that contains a sensor to detect when a needle has reached a synovial cavity of a patient to alert the operator that the needle is correctly placed in the synovial cavity.

In one aspect of the invention the sensor is a pressure sensor capable of detecting fluid flow resistance.

In another aspect of the invention the sensor is a motion sensor, such as an accelerometer, to detect the difference in the movement of the needle as it passes through material differing in viscosity.

The present invention further provides a medical apparatus for administering an intra articulatar injection or aspiration, the apparatus comprises:

a hollow piercing member through which fluid may pass, the piercing member having a first end adapted to pierce a patient, and a second end releasably secured to a body;

a sensing means, wherein upon entering a cavity of the patient the sensing means senses when the first end of the piercing member is in the cavity

The purpose of this apparatus is to remove the deterrent to wider adoption of the intra-articular procedures, such as Visco Supplementation, due to the difficulty in correct insertion of the needle by less experienced practitioners. The apparatus includes a device that will address to some degree the needle placement concerns, increasing the popularity of this procedure.

The present invention further provides a method for accurately positioning a piercing member, in the form of a needle in a knee cavity of a patient, the method utilises an apparatus as above described, the method comprises the steps of:

positioning the patient and orientating the knee appropriately;

identifying the needle entry point and needle size;

cleaning the area at the needle entry point;

attaching the needle to a body incorporating an inducer;

inducing a pulse in the apparatus;

inserting the needle slowly into the knee and monitoring the induced pulse;

determining when a first end of the needle enters the cavity as a result of changes in pulse;

dispensing a small volume of sensing fluid to open Synovial cavity as the needle tip enters the Synovial cavity;

detaching the inducer from the needle;

attaching a syringe and dispensing the required volume of fluid into the cavity; and

removing the needle once the fluid has been dispensed and dress needle entry point if required

The step of determining when the first end of the needle enters the cavity may be observed on a display unit which displays the read out from the sensing means in the apparatus

The method may also comprise the step of flushing the cavity before injecting the fluid. Preferably if flushing of the joint is required the piercing member of the apparatus is in the form of a cannula.

The above procedure can also be performed in relation to joints other than the knee joint

The method for flushing the cavity may comprise the steps of:

setting the required fluid volumes and dispensing Saline;

aspirating waste

The above two steps may need to be repeated until the joint is flushed

The method of flushing the joint may first comprise the step of aspirating any effusion

The present invention further provides a method for accurately positioning a piercing member, in the form of a needle in a knee cavity of a patient, the method utilises an apparatus as above described, the method comprises the steps of:

positioning the patient and orientating the knee appropriately;

identifying the needle entry point and needle size;

cleaning the area at the needle entry point;

inserting the needle slowly into the knee and causing a fluid to pass at a constant rate through the needle and into the patient;

measuring and monitoring the resistance experienced by the fluid;

determining when a first end of the needle enters the cavity as a result of changes in resistance

The fluid that is the sensing agent, in the sense, it transmits and conducts the pressure or actually the resistance changes to the transducer at the end of the fluid column in the device

The method may further comprise the steps of:

dispensing a small volume of sensing fluid to open Synovial cavity as the needle tip enters the Synovial cavity;

attaching a syringe and dispensing the required volume of fluid into the cavity; and

removing the needle once the fluid has been dispensed and dressing the needle entry point if required

The present invention further provides a method for accurately positioning a piercing member, in the form of a needle in a knee cavity of a patient, the method utilises an apparatus as above described, the method comprises the steps of:

inserting the needle slowly into the knee and causing a fluid to pass continuously through the needle and into the patient;

measuring and monitoring the resistance experienced by the fluid;

determining when a first end of the needle enters the cavity as a result of change in resistance

The present invention further provides a method for accurately positioning a piercing member, in the form of a needle in a knee cavity of a patient, the method utilises an apparatus as above described, the method comprises the steps of:

connecting the needle to a disposable holder with a transducer incorporated;

attaching semi rigid tubing to a syringe and filling with sterile saline;

locating the syringe in the driver and attaching the needle arrangement to the other end of the semi rigid tubing;

when the needle is positioned in the tissue commence needle travel;

when the needle is in the cavity disconnecting the needle and continuing with the supplementation procedure; and.

disposing of the syringe, tubing and transducer

The apparatus may also include a visual or audible signal to indicate to the operator when the needle end has entered the cavity

Obviously the aforementioned apparatus and method can be used in relation to the injection of any fluid into any region of the body. The scope of this invention also covers such applications.

### BRIEF DESCRIPTION OF THE DRAWING

The invention will be better understood by reference to the following description of several specific embodiments thereof as shown in the accompanying drawings in which:

FIG. 1 is a schematic of the anatomy of a knee joint;

FIG. 2 is a block diagram of a medical apparatus according to an embodiment of the invention;

FIG. 3 is a cross sectional schematic of a medical apparatus according to a first embodiment;

FIG. 4 is a perspective schematic view of FIG. 3;

FIG. 5a is a detailed cross sectional view of FIG. 4;

FIG. 5b is a schematic cross sectional view of a portion of a medical apparatus according to a second embodiment

FIG. 6 is a schematic cross sectional view of a portion of a medical apparatus according to a third embodiment;

FIG. 7 is a front perspective view of a display unit;

FIG. 8 is a rear perspective view of the display unit in FIG. 7;

FIG. 9 is a cross sectional view of the medical apparatus according to the third embodiment;

FIG. 10 is a schematic view of the display unit connected to fluid bags;

FIG. 11 is an image of the screen on the display unit;

FIGS. 12a, 12b and 12c represent pressure pulse response through an 18 gauge needle with either no obstruction or full obstruction at 5 Hz, 20 Hz and 35 Hz respectively;

FIGS. 13a, 13b and 13c is similar to FIGS. 12a, 12b and 12c but at 8 Hz, 12 Hz and 30 Hz;

FIG. 14a represents pressure pulse response through an 18 gauge needle with full obstruction at varying frequencies;

FIG. 14b is similar to FIG. 15a but with no obstruction;

FIG. 15 is a schematic of a medical apparatus according to a fourth embodiment; and

FIG. 16 is an example of a syringe driver which may be used in the embodiments of the invention.

### BEST MODE(S) FOR CARRYING OUT THE INVENTION

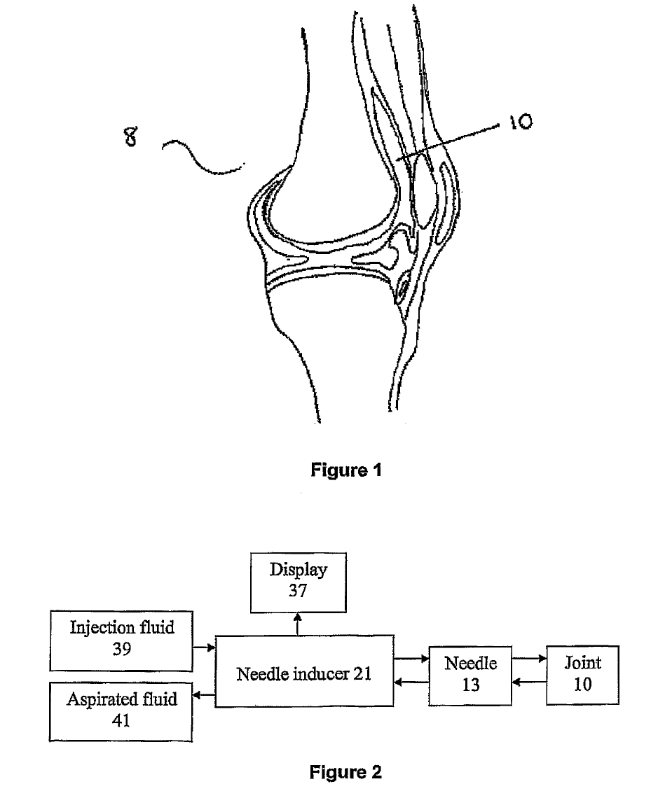

FIG. 1 is a schematic of the anatomy of a knee joint 8. Referring to FIGS. 1 and 2, the invention according to the first embodiment is in the form of a medical apparatus 11 for performing Visco supplementation. FIG. 2 is a block diagram of a medical apparatus 11 according to an embodiment of the invention. The apparatus 11 comprises a hollow piercing member, in the form of a needle or cannula 13 through which a fluid may pass. The needle/cannula 13 has a first end 15 adapted to pierce the skin of a patient, and a second end 17 releasably secured to a body 19. The body 19 incorporates an inducer 21 to induce pulses within the apparatus 11

The apparatus 11 also comprises a sensing means 23 wherein upon entering a cavity of the patient the sensing means 23 senses pulse differentials which result when the first end 15 of the needle/cannula 13 enters the cavity. Such as the synovial joint cavity 10 shown in FIG. 1.

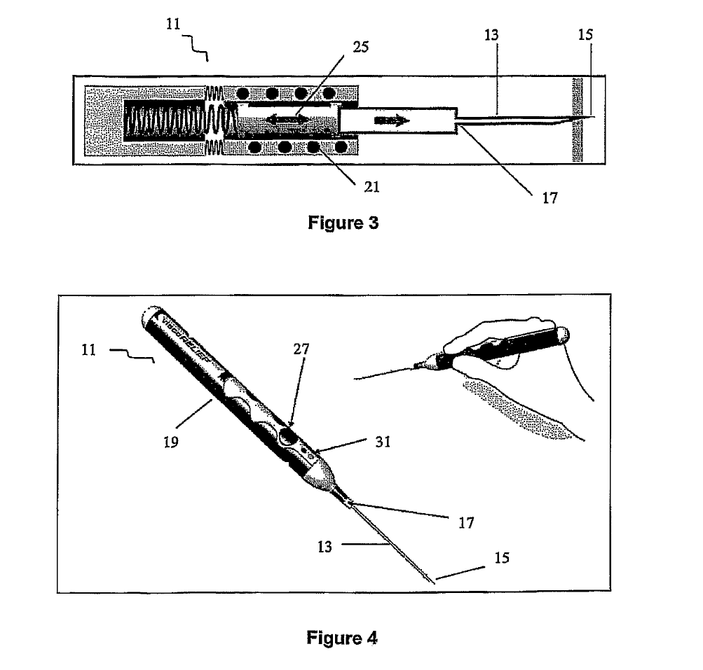

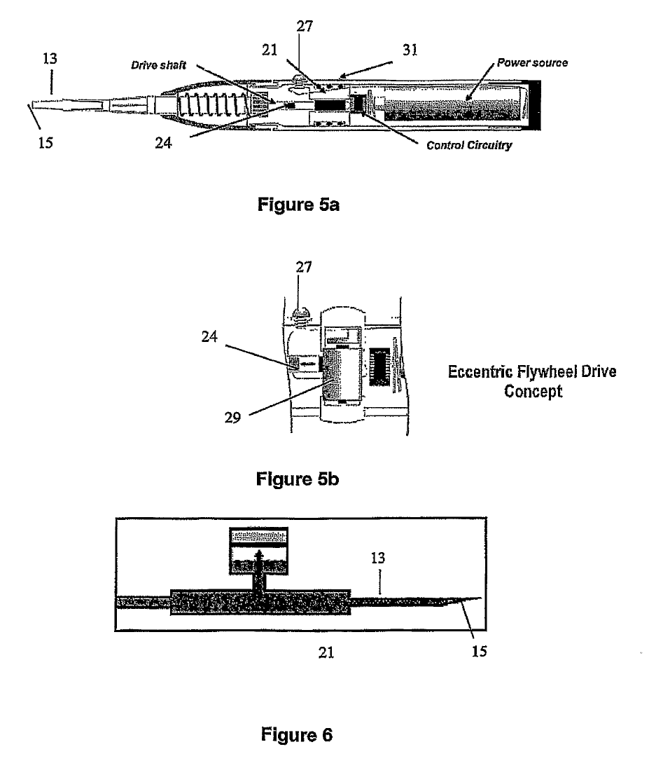

A first embodiment of the invention is shown in FIGS. 3, 4, and 5a. In this embodiment the pulse that is generated by the inducer is a force pulse and is generated by a mass 25 supported in the confines of the body 19. The mass 25 is movable in a forward and rear direction relative to the body 19. When activated by the operation of the power switch the mass 25 is caused to oscillate causing force pulses to act along the needle/cannula 13. The mass 25 is caused to move as a result of electro-magnetism created within a solenoid coil 21, as best shown in FIG. 5a.

According to a second embodiment, the force pulse is generated by eccentrically mounting a fly wheel 29 within the body 19, as shown in FIG. 5b.

In both the first and second embodiments the force pulse is generated by the inertia effects of the mass 25 and flywheel 29 respectively. When the first end 15 of the needle/cannula 13 enters a region of different viscosity such as the cavity, the change in inertial kickback is experienced. This change provides a physical indication to the operator that the first end 15 is in the cavity. The sensing means 23 in the form of an accelerometer 24 also senses the change and a visual indicator 31 on the body 19 indicates to the operator that the cavity has been entered.

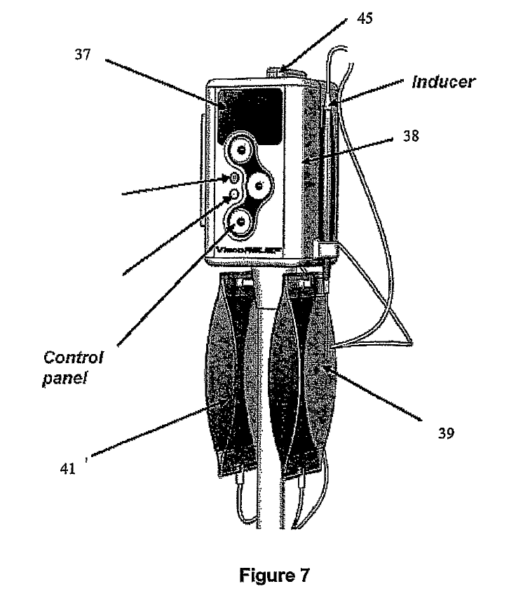

Referring to FIGS. 7 to 12, a third embodiment of the invention is shown. In this embodiment the pulses are in the form of pressure pulses which are induced in the fluid travelling through a tube 33 which is in fluid communication with the needle/cannula 13. The pressure pulses form at a known frequency and pressure.

The sensing means 23 in this embodiment is in the form of a transducer 35 which measures the change in resistance at the first end 15 of the needle/cannula 13.

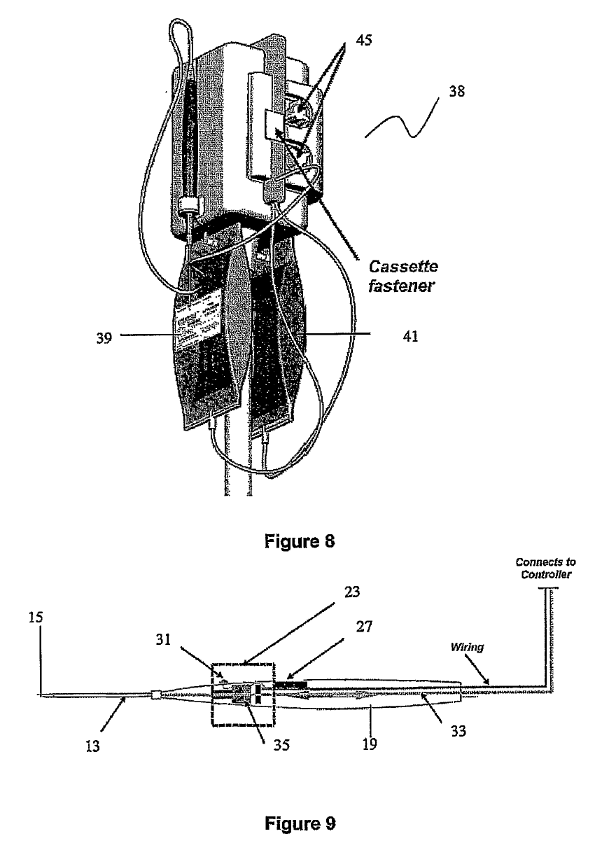

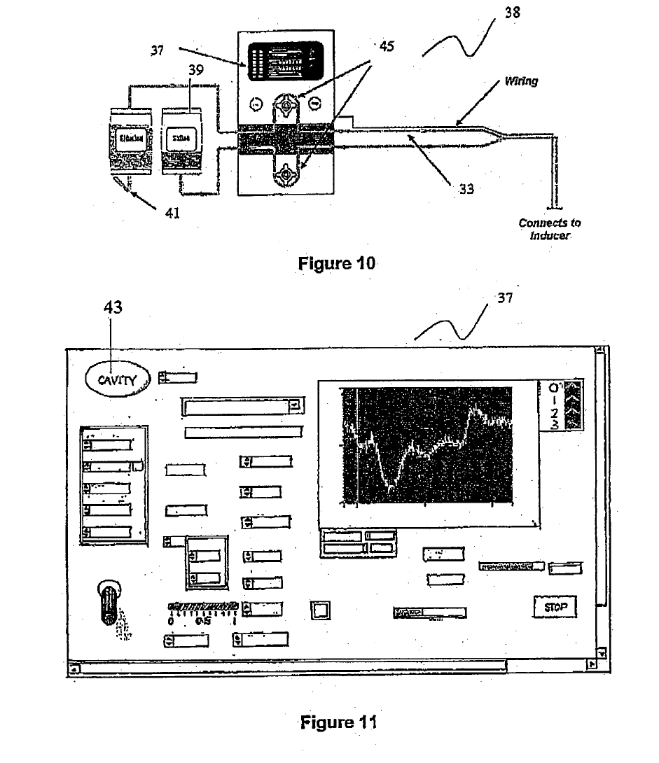

Referring to FIGS. 9 and 10, the apparatus 11 is connected to a display unit 37, which incorporates a pump unit 38, a saline bag 39 and an effusion bag 41. The display of the display unit 37 may take many forms, an example of which is shown in FIG. 11. On this display is an indicator lamp 43 which is highlighted when first end 15 enters the cavity.

The pump unit 38 comprises two peristaltic pumps 45, each designated to either move fluid out the saline bag 39 or into the effusion bag 41 to ensure there is no cross contamination

The restriction to flow of various fluids such as saline through a needle as the needle passes through various tissue types was characterised to provide an understanding of the flow properties. This was achieved by:

measuring flow resistance/penetration profile through various tissue compositions with feed rate as a parameter;

measuring fluid flow/pressure profile into tissue with needle stalled; and

measure fluid flow/pressure profile into the synovial cavity.

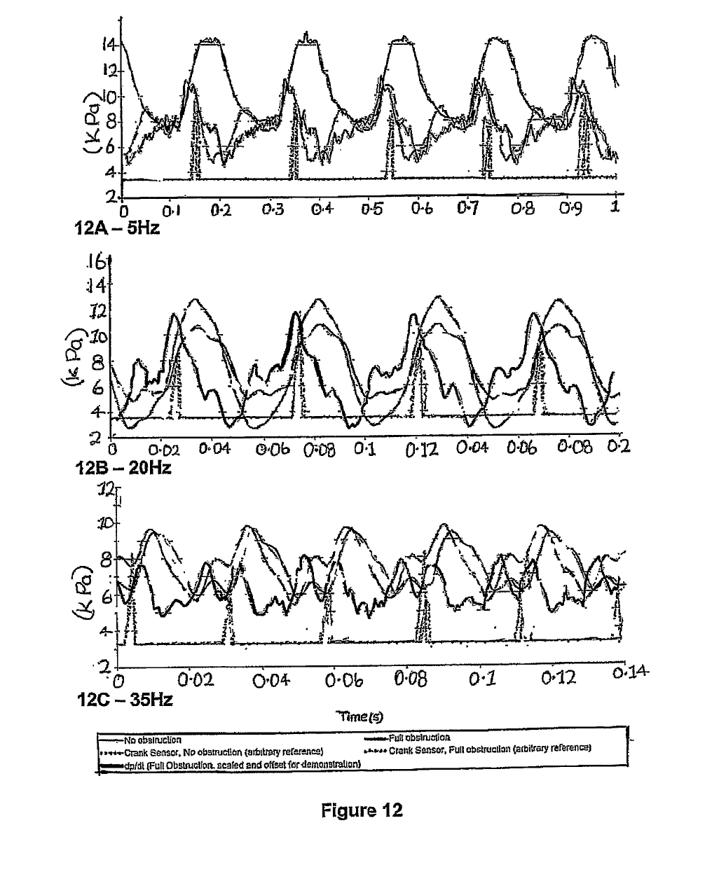

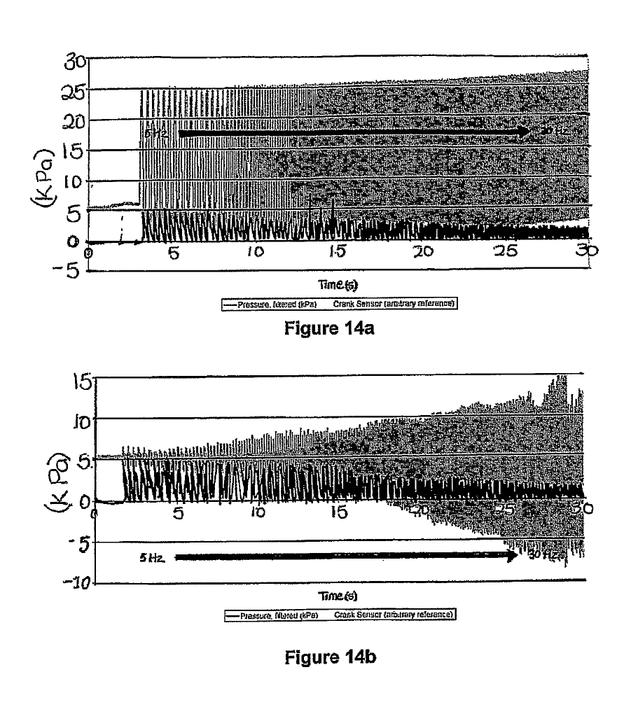

The focus of the flow sensing investigative tests has been on the detection of the transition between an obstructed and open needle tip. Following initial discrete pulse testing, tests were conducted with a range of continuous pulse frequencies and injection flow rates. Tests were then confined to 10 μl pulse volumes at 5, 20 and 35 Hz. The results can be seen in FIGS. 12, 13 and 14. A phase shift between the obstructed and unobstructed needle tip was observable at all three frequencies.

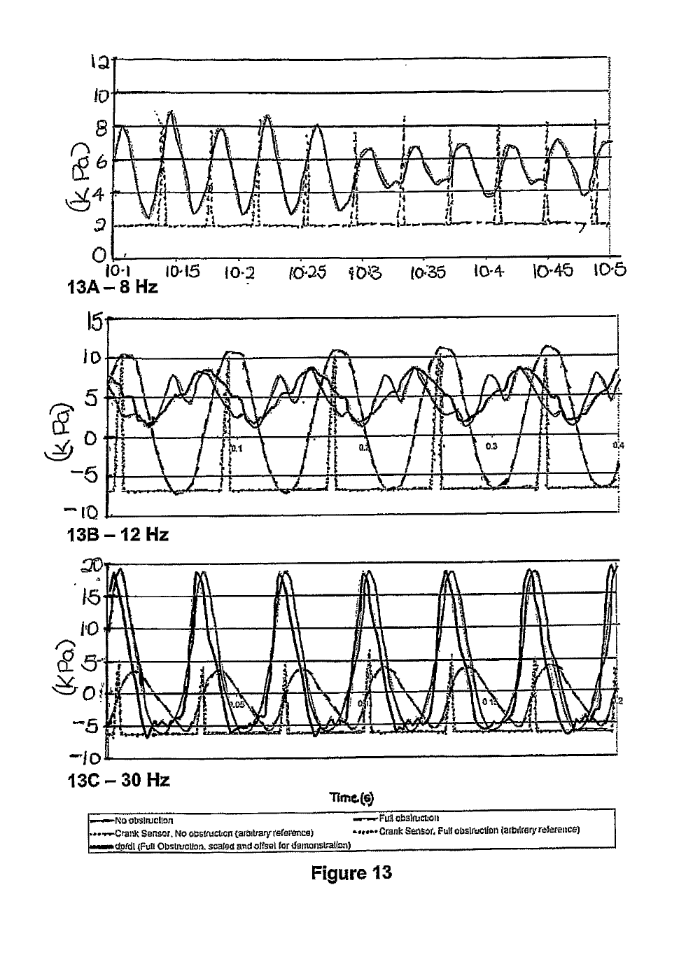

At 8 Hz pulsing rate every second pulse is dropped because of processing time effective pulse rate is 4 Hz, FIG. 13A. At this pulse rate the controller consistently detects transition between closed needle tip and open needle tip. At this pulse rate the controller was able to consistently detect the difference between the test specimen and air.

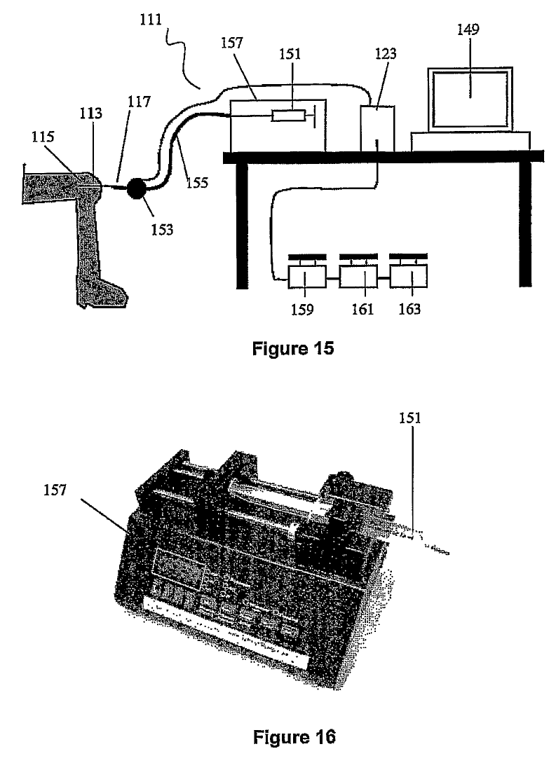

Referring to FIG. 15, the invention according to the fourth embodiment is in the form of a medical apparatus 111 for performing Visco supplementation. The apparatus 111 comprises a hollow piercing member, in the form of a needle or cannula 113 through which a fluid may pass. The needle/cannula 113 has a first end 115 adapted to pierce the skin of a patient, and a second end 117 releasably secured to a syringe 151 using tubing 155.

The apparatus 111 also comprises a sensing means 123 wherein upon entering a cavity of the patient the sensor means 123 senses changes in resistance which result as the needle passes through the patient's tissue. A noticeable change in resistance is measured when the first end 115 of the needle/cannula 113 enters the cavity.

The apparatus 111 is used to determine when the needle is correctly located in a cavity of the body. The fourth embodiment requires a constant flow of fluid to be supplied to the needle 113 with the sensing means 123 measuring the changes in resistance as the needle is inserted deeper into the patient.

The sensing means 123 may incorporate a data logging system to collect the data at the needle end 115 and display on display monitor 149 to provide a visible representation of the measurements being taken. The data logging system may record the pressure profile and the insertion rate.

The apparatus 111 is used in the following manner:

a. Connect the needle 113 to a disposable holder 153 with transducer incorporated (not shown);

b. Attach semi rigid tubing 155 to the syringe 151 and fill with sterile saline;

c. Locate the syringe 151 in a syringe driver 157 and attach the needle arrangement to the other end of the semi rigid tubing 155;

d. When the needle 113 is inserted into the tissue press a first foot-switch 159;

e. When insertion (needle travel) commences push a second foot-switch 161;

f. When insertion finishes (cavity located) push a third foot switch 163;

The foot switches are optional as they are only used when there is a requirement for collecting information regarding the changes in resistance as the needle passes through the knee joint.

g. Disconnect needle 113 and continue with either elution or supplementation procedure.

h. Dispose of syringe, tubing and transducer

The syringe driver 157 provides a constant force to the syringe 151 so that fluid flow is constant.

One procedure the current invention has application is Visco Supplementation. Visco Supplementation is a procedure for treating patients suffering pain due to osteoarthritis of the knee. The procedure involves the administration of synthetic synovial fluid via a syringe and needle to the synovial capsule within the patients' knee joint.

The development of the current invention can assist the practitioner in correctly identifying when the needle tip is located within a cavity of the body.

Modifications and variations such as would be apparent to the skilled addressee are considered to fall within the scope of the present invention.

Throughout the specification, unless the context requires otherwise, the word “comprise” or variations such as “comprises” or “comprising”, will be understood to imply the inclusion of a stated integer or group of integers but not the exclusion of any other integer or group of integers.

The claims defining the invention are as follows:

1. A medical apparatus for an intra-articular injection or aspiration, the apparatus comprises:
a hollow piercing member through which fluid may pass, the piercing member having a first end adapted to pierce a patient, and a second end releasably secured to a body;
a sensing means, wherein upon entering a cavity of the patient the sensor means senses the change in resistance at the first end of the piercing member which results when the first end of the piercing member enters the cavity.

2. The medical apparatus according to claim 1 wherein the piercing member is in the form of a needle or cannula.

3. The medical apparatus according to claim 1 or 2 wherein the sensor means is in the form of a pressure sensor capable of detecting fluid flow resistance.

4. The medical apparatus according to claim wherein 3 the sensing means senses the resistance to fluid flow relative to the first end of the piercing member, providing an indication to an operator that the first end of the piercing member is in the cavity.

5. The medical apparatus according to any one of the preceding claims wherein the fluid is injected into the patient at a constant rate.

6. The medical apparatus according to claim 1, 2, 3 or 4 wherein the inducer comprises a pulse mechanism for delivering pulses to the piercing member, whereby the pulses are measured by the sensor means.

7. The medical apparatus according to claim 6 wherein the pulse mechanism for providing pulses comprises a tube within a body of the apparatus, wherein the tube is in fluid communication with the piercing member and contains a fluid therein which is caused to pulse at a predetermined repetition rate and pressure.

8. The medical apparatus according to claim 7 wherein the fluid is a hyaluronic acid supplement.

9. The medical apparatus according to any one of the preceding claims wherein the sensor means comprises a transducer to measure changes in pressure within the piercing member.

10. The medical apparatus according to claim 9 wherein a change in resistance at the first end of the piercing member is detected by a threshold change in the transducer.

11. The medical apparatus according to claim 9 or 10 wherein the apparatus is connected to a display unit which displays the measurements taken from the transducer and therefore indicates to the operator when the first end of the piercing member enters different regions of the joint.

12. The medical apparatus according to claim 1, 2, 3 or 4 wherein the sensor is a motion sensor to detect the difference in the movement of the piercing member as it passes through material differing in viscosity.

13. The medical apparatus according to claim 12 wherein the sensor means amplifies the change in reaction force as the first end moves through different tissue.

14. The medical apparatus according to claim 12 or 13 wherein the pulse mechanism delivers force pulses to the piercing member, whereby the pulses forwardly induce the piercing member whilst the piercing member is being pushed by the operator.

15. The medical apparatus according to claim 12, 13 or 14 wherein the pulse mechanism is in the form of a mass adapted to move relative to a body in a forward and back direction according to the movement of the apparatus wherein the movement of the mass and its inertial effects deliver the force pulses, inducing the piercing member forward.

16. The medical apparatus according to claim 15 wherein the mass is incorporated within the body.

17. The medical apparatus according to any one of claims 12 to 16 wherein the sensor means senses a reduction in inertial kickback through the body of the apparatus.

18. The medical apparatus according to claim 12, 13 or 14 wherein the pulse mechanism induces force pulses using an eccentrically mounted flywheel.

19. The medical apparatus according to any one of claims 7 to 18 wherein the body is removed from the piercing member and replaced by a syringe.

20. The medical apparatus according to any one of the preceding claims wherein the apparatus comprises a flushing device to clean the cavity into which the piercing member is inserted.

21. The medical apparatus according to claim 20 wherein the flushing device is incorporated with the pulse mechanism.

22. The medical apparatus according to any one of the preceding claims wherein the apparatus comprises a visual indication which is highlighted or an audible signal which is sounded when the sensor means senses that the first end of the piercing member has entered the cavity.

23. A needle device that contains a sensor to detect when a needle has reached a synovial cavity of a patient to alert the operator that the needle is correctly placed in the synovial cavity.

24. The medical apparatus according to claim 23 wherein the sensor is a pressure sensor capable of detecting fluid flow resistance.

25. The medical apparatus according to claim 23 wherein the sensor is a motion sensor, such as an accelerometer, to detect the difference in the movement of the needle as it passes through material differing in viscosity.

26. A medical apparatus for administering an intra articulatar injection or aspiration, the apparatus comprises:
a hollow piercing member through which fluid may pass, the piercing member having a first end adapted to pierce a patient, and a second end releasably secured to a body;
a sensing means, wherein upon entering a cavity of the patient the sensor means senses when the first end of the piercing member is in the cavity.

27. A method for accurately positioning a piercing member, in the form of a needle in a knee cavity of a patient, the method utilises an apparatus as above described, the method comprises the steps of:
positioning the patient and orientating the knee appropriately;
identifying the needle entry point and needle size;
cleaning the area at the needle entry point;
attaching the needle to a body incorporating an inducer;
inducing a pulse in the apparatus;
inserting the needle slowly into the knee and monitoring the induced pulse;
determining when a first end of the needle enters the cavity as a result of changes in pulse;
dispensing a small volume of sensing fluid to open Synovial cavity as the needle tip enters the Synovial cavity;
detaching the inducer from the needle;
attaching a syringe and dispensing the required volume of fluid into the cavity; and
removing needle once the fluid has been dispensed and dress needle entry point if required.

28. The method according to claim 27 wherein the step of determining when the first end of the needle enters the cavity is observed on a display unit which displays the read out from the sensor means in the apparatus.

29. The method according to claim 27 or 28 further comprising the step of flushing the cavity before injecting the fluid.

30. The method according to claim 29 whereby when flushing the joint the piercing member of the apparatus is in the form of a cannula.

31. The method according to claim 29 or 30 wherein flushing is achieved by:
a. setting the required fluid volumes and dispensing Saline;
b. aspirating waste;

32. The method according to claim 31 first comprises the step of aspirating any effusion before dispensing the Saline.

33. A method for accurately positioning a piercing member, in the form of a needle in a knee cavity of a patient, the method utilises an apparatus as above described, the method comprises the steps of:
positioning the patient and orientating the knee appropriately;
identifying the needle entry point and needle size;
cleaning the area at the needle entry point;
inserting the needle slowly into the knee and causing a fluid to pass at a constant rate through the needle and into the patient;
measuring and monitoring the resistance experienced by the fluid;
determining when a first end of the needle enters the cavity as a result of changes in resistance;

34. The method according to claim 33 further comprising the step of:
dispensing a small volume of sensing fluid to open Synovial cavity as the needle tip enters the Synovial cavity;
attaching a syringe and dispensing the required volume of fluid into the cavity; and
removing needle once the fluid has been dispensed and dress needle entry point if required.

35. A method for accurately positioning a piercing member, in the form of a needle in a knee cavity of a patient, the method utilises an apparatus as above described, the method comprises the steps of:
inserting the needle slowly into the knee and causing a fluid to pass continuously through the needle and into the patient;
measuring and monitoring the resistance experienced by the fluid;
determining when a first end of the needle enters the cavity as a result of change in resistance;

36. A method for accurately positioning a piercing member, in the form of a needle in a knee cavity of a patient, the method utilises an apparatus as above described, the method comprises the steps of:
connecting the needle to a disposable holder with a transducer incorporated;
attaching semi rigid tubing to a syringe and fill with sterile saline;
locating the syringe in the driver and attaching the needle arrangement to the other end of the semi rigid tubing;
when the needle is positioned in the tissue commence needle travel;
when the needle is in the cavity disconnecting the needle and continuing with supplementation procedure; and.
disposing of syringe, tubing and transducer.

37. A medical apparatus for an intra-articular injection or aspiration, the apparatus comprises:
a hollow piercing member through which fluid passes at a constant rate, the piercing member having a first end adapted to pierce a patient, and a second end releasably secured to a body;
a sensing means,
wherein upon entering a cavity of the patient fluid is injected into the patient and the sensor means senses the change in resistance which results when the first end of the piercing member enters the cavity.

38. The apparatus according to claim 37 wherein the sensing means is in the form of a pressure sensor.

39. The apparatus according to claim 37 or 38 wherein the sensing means comprises a data logging system that records the pressure profile during the procedure.

40. A method for accurately positioning a piercing member as substantially herein described.

41. A medical apparatus for an intra-articular injection or aspiration as substantially herein described with reference to the drawings.
